(19) United States
(12) Patent Application Publication
(10) Pub. No.: US 2024/0424272 A1
(43) Pub. Date: Dec. 26, 2024

(54) MICRONEEDLE DEVICE AND MICRONEEDLE SYSTEM USING THE SAME

(71) Applicant: CURIOSIS CO., LTD, Seoul (KR)

(72) Inventors: Chihyoung Shim, Seoul (KR); Kichan Kim, Seoul (KR); Kyuntae Kim, Seoul (KR); Jihoon Shin, Seoul (KR); Heedon Ahn, Seoul (KR); Hoyoung Yun, Seoul (KR)

(73) Assignee: CURIOSIS CO., LTD, Seoul (KR)

(21) Appl. No.: 18/748,226

(22) Filed: Jun. 20, 2024

(30) Foreign Application Priority Data
Jun. 20, 2023 (KR) 10-2023-0078660
Jun. 19, 2024 (KR) 10-2024-0079947

(51) Int. Cl. A61M 37/00; A61N 1/40; A61N 5/06; A61N 5/067

(52) U.S. Cl. CPC A61M 37/0015; A61N 1/40; A61N 5/062; A61N 5/067; A61M 2037/0007; A61M 2037/0023; A61M 2205/0238; A61N 2005/063; A61N 2005/0651

(57) ABSTRACT

According to an example embodiment of the present disclosure, there is provided a microneedle device for forming fine holes in a skin to inject effective ingredients applied on the skin to the skin via the fine holes. The microneedle device According to an example embodiment of the present disclosure may include a housing, a driver configured to generate a driving force for operating a plurality of microneedles, a needle assembly provided with the plurality of microneedles, a controller configured to control an operation of the microneedle device, and a radio-frequency (RF) energy transfer module configured to transfer radio frequency energy to the skin. According to an example embodiment of the present disclosure, the RF energy transfer module may include an RF positive electrode and an RF negative electrode, and one side of the RF energy transfer module may be configured to be exposed forward of a microneedle device.

### CROSS-REFERENCE TO RELATED APPLICATIONS

This application claims priority to Korean Patent Application No. 10-2023-0078660 filed on Jun. 20, 2023 and Korean Patent Application No. 10-2024-0079947 filed on Jun. 19, 2024, the entire contents of which are herein incorporated by reference.

### TECHNICAL FIELD

The present disclosure relates to a microneedle device and a microneedle system using the same, and more particularly to a microneedle device configured such that transferring radio-frequency (RF) energy and/or a light irradiation are performed while forming fine holes in a skin with microneedles and subsequently injecting effective ingredients such as effective agents to the skin via the fine holes, and a microneedle system using the same.

### BACKGROUND

A health condition of a skin has a large impact on the appearance. Thus, in recent years, various methods have been developed for the purpose of lightening the skin, improving wrinkles, moisturizing the skin, increasing resilience of the skin, and the like, in addition to the purpose of treating a skin disease.

As is well known, the human skin may be roughly classified into an epidermal layer (epidermis), a dermal layer (dermis) and a fat layer. Among these, the dermal layer occupying a substantial portion of the skin is composed of a papillary layer and a plexiform layer. Capillaries, lymphatic tubes and the like are located in the papillary layer. Collagens as collagenous fibers associated with wrinkles in the skin, elastin as elastic fibers that give elasticity to the skin, a substrate and the like are contained in the plexiform layer.

On the other hand, a visual condition of the skin is greatly dependent on a health status of the dermal layer. Accordingly, various methods implemented for improving the condition of the skin are often targeted for the dermal layer.

As a method which has been widely used recently for skin care, there is a treatment method of transferring the RF energy to the skin to promote strengthening of the collagen fibers and apply resilience to the skin.

In general, the treatment using the RF energy is performed in a non-invasive manner in which the RF energy is transferred inward of the skin by applying radio frequency using an electrode contacted to the skin.

However, in the case of the treatment using the RF energy in the related art, a large amount of energy loss may occur when the RF energy is transferred to the dermal layer via the epidermal layer. Because of this, a large amount of energy needs to be applied to the surface of the skin in order to transfer the RF energy to a deep position in the skin. This may cause matters such as burns on the surface of the skin.

As another method which has been widely used for skin care, there is a light irradiation method which irradiates the skin with light to deliver light energy to the skin, thereby treating and improving the skin.

In general, such a light irradiation method may be classified into a method of irradiating a laser with concentrated energy to burn the surface of the skin and a method of penetrating light of a specific wavelength from a light emitting device (LED) into the skin to treat and improve the skin treatment.

In the case of the light irradiation method using the laser, the laser may penetrate into the skin and transfer the energy inward of the skin. However, this method needs to irradiate a high energy light to the skin, which may cause matters such as burns on the surface of the skin.

On the other hand, in the case of the method using the LED light, the concentration of the light is difficult so that not only the intensity of the light energy is weak, but also reflection significantly occurs at the surface of the skin. As a result, sufficient light may not be penetrated to an actual desired depth, which makes it difficult to obtain desired treatment and improvement effects of the skin.

### SUMMARY

The present disclosure was made for the purpose of solving the above matters, and the present disclosure is for the purpose of a microneedle device configured such that transferring radio-frequency (RF) energy and/or a light irradiation are performed while forming fine holes in a skin with microneedles and subsequently injecting effective ingredients such as effective agents to the skin via the fine holes, and a microneedle system using the same.

Representative configurations of the present disclosure to achieve the above aspects are described below.

According to an example embodiment of the present disclosure, a microneedle device for forming fine holes in a skin to inject effective ingredients applied on the skin to the skin via the fine holes may be provided. The microneedle device according to an example embodiment of the present disclosure may include a housing, a driver configured to generate a driving force for operating a plurality of microneedles, a needle assembly provided with the plurality of microneedles, a controller configured to control an operation of the microneedle device, and a radio-frequency (RF) energy transfer module configured to transfer radio frequency energy to the skin. According to an example embodiment of the present disclosure, the RF energy transfer module may include an RF positive electrode and an RF negative electrode, and one side of the RF energy transfer module may be configured to be exposed forward of a microneedle device.

In an aspect, the RF energy transfer module may include a contact film at a front end portion of the needle assembly, and one side of the RF positive electrode and one side of the RF negative electrode may be configured to be electrically connected to the contact film.

In an aspect, in a state in which the plurality of microneedles is inserted into the skin to form the fine holes and effective agents are injected to the skin via the fine holes, the radio frequency energy may be transferred to the skin by the RF positive electrode and the RF negative electrode.

According to another example embodiment of the present disclosure, a microneedle device for forming fine holes in a skin to inject effective ingredients applied on the skin to the skin via the fine holes may be provided. The microneedle device According to an example embodiment of the present disclosure may include a housing, a driver configured to generate a driving force for operating a plurality of microneedles, a needle assembly provided with the plurality of microneedles, a controller configured to control an operation of the microneedle device, and a light irradiation module configured to irradiate the skin with light via the plurality of microneedles. According to an example embodiment of the present disclosure, each of the plurality of microneedles may be formed in a tubular structure having a through-hole at a central portion thereof, and the light irradiation module may be configured to irradiate the light forward of the microneedle device via the through-hole formed in the central portion of each of the plurality of microneedles.

In an aspect, an inner circumferential surface of the through-hole formed in the central portion of each of the plurality of microneedles may be coated with a material having a relatively high reflectance.

In an aspect, an optical fiber may be inserted into the through-hole formed in the central portion of each of the plurality of microneedles.

In an aspect, the light irradiation module may be configured to irradiate the skin with the light in a state in which the plurality of microneedles is inserted into the skin.

In an aspect, a light source of the light irradiation module may be configured to emit light of a plurality of wavelengths.

In an aspect, the light irradiation module may be configured to irradiate light of different wavelengths corresponding to depths at which the plurality of microneedles is inserted into the skin.

In an aspect, the microneedle device may further include a pump unit configured to generate compressed air and supply the compressed air into the needle assembly, and a valve configured to open and close a pneumatic hose provided between the pump unit and the needle assembly.

In an aspect, the valve may be open and closed under a control of the controller. When a tip end of the needle assembly is brought into close contact with the skin, an interior of the needle assembly may be closed to form a closed space. When the valve is open, the compressed air may be supplied from the pump unit to the interior of the needle assembly so that a positive pressure is generated in the closed space of the needle assembly.

In an aspect, the controller may be configured to control the opening and closing of the valve based on information input from a user.

In an aspect, the controller may be configured to control a needling operation in which the plurality of microneedles is inserted into the skin and subsequently, return to original positions thereof. Further, the controller may be configured to control the microneedle device in a stacking mode in which a penetration depth is set for each needling operation.

In an aspect, the stacking mode may be set such that the needling operation is repeated twice or more times at a same skin position or at different time intervals. In the stacking mode, the needling operation may be performed at a same penetration depth or different penetration depths.

According to another example embodiment of the present disclosure, there may be provided a microneedle system including the aforementioned microneedle device and a display device which is in communication with the controller of the microneedle device, and configured to provide a user interface for receiving setting information about the operation of the microneedle device from a user and transmit the setting information input from the user to the controller of the microneedle device.

Further, the microneedle device and the microneedle system using the same according to the present disclosure may further include other additional configurations without departing from the technical sprit of the present disclosure.

A microneedle device according to an example embodiment of the present disclosure is configured to form fine holes in the skin by microneedles and subsequently transfer RF energy into the skin via the fine holes. This makes it possible to transfer a sufficient intensity of RF energy to a deep position in the skin even with less energy, thereby improving skin regeneration and treatment effects without causing matters such as burns on the skin.

Further, a microneedle device according to an example embodiment of the present disclosure is configured to form fine holes in the skin with microneedles and subsequently irradiate light into the skin via the fine holes. This makes it possible to stably irradiate the light into the skin without damaging the surface of the skin and stably perform a light irradiation.

In addition, a microneedle device according to an example embodiment of the present disclosure is configured to irradiate light of necessary wavelengths according to depths of fine holes formed by microneedles. This makes it possible to suitably irradiate the light of necessary wavelengths to various positions inside the skin, thereby efficiently perform skin reproduction and treatment functions.

Further, a microneedle device according to an example embodiment of the present disclosure is configured to inject effective agents, which are encapsulated with a substance whose morphology changes at a specific wavelength (or specific temperature), using microneedles, and subsequently irradiate energy of the specific wavelength (or irradiate energy to raise the temperature of the encapsulated substance) to work the effective agents in a state in which the microneedles has penetrated by a treatment depth. This makes it possible to perform a precision treatment using the effective agents that act only at a specific lesion or at a specific skin depth.

Further, a microneedle device according to an example embodiment of the present disclosure is configured to inject effective ingredients such as effective agents applied on the skin into the skin using compressed air while alternately generating a negative pressure and a positive pressure with a pump unit and a valve. This makes it possible to more easily penetrate the effective ingredients such as the effective agents into the skin in a more stable manner.

Further, a microneedle device according to an example embodiment of the present disclosure is configured such that microneedles repeatedly penetrates into the skin multiple times in a stacking mode. This makes it possible to treat several layers in the skin (for example, a papillary layer, a plexiform layer and the like) at once by a mechanism of microneedling and the injection of effective ingredients (effective agents and the like).

### BRIEF DESCRIPTION OF THE DRAWINGS

FIG. 1 schematically illustrates an overall configuration of a microneedle system according to an example embodiment of the present disclosure.

FIG. 2 exemplarily illustrates a basic structure of a microneedle device according to an example embodiment of the present disclosure.

FIG. 3 exemplarily illustrates an internal structure of the microneedle device illustrated in FIG. 2.

FIG. 4 exemplarily illustrates a cross-sectional structure of a needle assembly provided in the microneedle device illustrated in FIG. 2.

FIGS. 5A and 5B exemplarily illustrate a front structure of the needle assembly provided in the microneedle device illustrated in FIG. 2.

FIG. 6 schematically illustrates a principle of injecting effective ingredients, such as effective agents into a skin by microneedles of a hollow structure in the microneedle device according to an example embodiment of the present disclosure.

FIG. 7 exemplarily illustrates a structure of the needle assembly to which a radio-frequency (RF) energy transfer module is added, and a principle in which RF energy is transferred into the skin by the RF energy transfer module.

FIGS. 8A and 8B exemplarily illustrate a structure of a front end portion of the needle assembly illustrated in FIG. 7.

FIGS. 9A and 9B exemplarily illustrate a structure of a microneedle in which an optical fiber is inserted into a central portion of the microneedle.

FIG. 10 exemplarily illustrates a principle in which light is irradiated to the skin through the microneedle illustrated in FIG. 9.

FIG. 11 exemplarily illustrates how the microneedle device according to an example embodiment of the present disclosure operates.

FIGS. 12A and 12B and FIG. 13 exemplarily illustrate a principle of a needling operation of the microneedle device according to an example embodiment of the present disclosure (FIGS. 12A and 12B exemplarily illustrate the principle of the needling operation of the microneedle device using a hollow microneedle, and FIG. 13 exemplarily illustrates the principle of the needling operation of the microneedle device using a solid microneedle).

FIG. 14 exemplarily illustrates how a pump unit and a valve operate to generate a negative pressure and a positive pressure in the microneedle device according to an example embodiment of the present disclosure.

FIGS. 15A and 15B schematically illustrate a state in which the microneedle is inserted into the skin in the microneedle device according to an example embodiment of the present disclosure [FIG. 15A illustrates a state in which a final penetration depth is set to 4 millimeter (mm) and the microneedle is penetrated by a depth of 3 mm, and FIG. 15B illustrates a state in which the microneedle is penetrated by 4 mm, which is the final penetration depth, and subsequently, is retracted by 1 mm]

FIG. 16 exemplarily illustrates a method of injecting effective ingredients such as effective agents into the skin with the microneedle device according to an example embodiment of the present disclosure.

FIGS. 17 and 18 exemplarily illustrate a user interface for receiving setting information input from a user to implement a stacking mode operation in the microneedle system according to an example embodiment of the present disclosure [FIG. 17 illustrates an interface for receiving time interval information input from the user, and FIG. 18 illustrates an interface for receiving information about a penetration depth for each needling operation from the user].

### DETAILED DESCRIPTION

Example embodiments of the present disclosure described herein are exemplified for the purpose of describing the technical spirit of the present disclosure. The scope of the claims according to the present disclosure is not limited to the example embodiments described below or to the detailed descriptions of these example embodiments.

Unless otherwise defined, all technical and scientific terms used herein have the same meaning commonly understood by those skilled in the art to which the present disclosure pertains. All terms used herein are selected for the purpose of more clearly describing the present disclosure and not limiting the scope of the present disclosure defined by appended claims.

Unless the phrase or sentence clearly indicates otherwise, terms “comprising” “including” “having” and the like used herein should be construed as open-ended terms encompassing the possibility of including other example embodiments.)

The terms “front,” “tip end,” and the like used herein mean a direction in which a skin is located relative to a microneedle device, and the terms “back,” “rear end,” and the like used herein mean a direction opposite the direction.

Further, the term “skin” used herein should be understood as the entire skin including a scalp, and a microneedle device of the present disclosure may also be used to inject effective ingredients into the scalp.

The singular form described herein may include the plural form unless the context clearly dictates otherwise, and this is equally applied to the singular form set forth in the claims.

Throughout the present specification, when a constituent element is referred to as being “positioned” at or “formed” on one side of another constituent element, the constituent element may be in direct contact with or directly formed on the one side of another constituent element, or may be positioned at or formed on another constituent element by intervening yet another constituent element therebetween.

In addition, the terms such as “-part,” “-module,” “unit” and the like used herein may refer to a part that performs at least one function or operation, which may be realized as hardware or software, or may be realized as a combination of hardware and software.

The expression “configured (or set) to-” described herein may be alternatively expressed to, for example, “suitable for-,” “having the capacity to-,” “designed to-,” “adapted to-,” “made to-,” “capable of-,” or the like depending on situations. The expression “configured (or set) to-” may not necessarily mean only “specifically designed to-” in hardware. Alternatively, in some circumstances, the expression “system configured to-” may mean that the system is “capable of-” together with other apparatus(es) or constituent element(s). The phrase “processor configured (or set) to perform A, B and C” may mean, for example, a dedicated processor (for example, embedded process) configured to perform a respective operation, or a generic-purpose processor (for example, a central processing unit (CPU) or an application processor) capable of performing respective operations by executing one or more software programs stored in a memory.

Further, although the terms including ordinal numbers such as a first, a second and the like used herein may be used to describe various constituent elements, the order or importance of the respective constituent elements is not limited by these terms unless otherwise defined.

Hereinafter, example embodiments of the present disclosure will be described in detail with reference to the accompanying drawings at such an extent that they may be readily practiced by those ordinary skilled in the art. In the accompanying drawings, the same reference numerals are assigned to the same or corresponding components. Further, in the following descriptions of the example embodiments, duplicate descriptions of the same or corresponding constituent elements may be omitted. However, even though a description of a constituent element is omitted, such a constituent element is not intended to be excluded in any example embodiment.

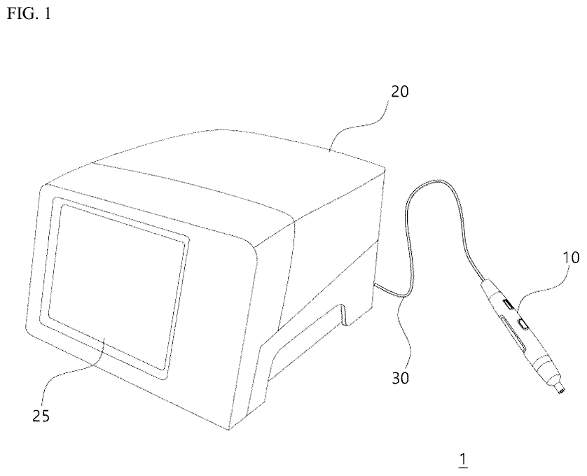

Referring to FIG. 1, there is exemplarily illustrated a schematic view of a microneedle system 1 according to an example embodiment of the present disclosure.

As illustrated in FIG. 1, the microneedle system 1 may include a microneedle device (or handpiece) 10, a display device 20, a cable 30 which connects the microneedle device 10 and the display device 20, and the like.

The microneedle device 10 is a major constituent element of the present disclosure, which will be described in detail below.

The display device 20 may include a user interface 25 (for example, a touch screen) to receive setting information about an operation of the microneedle device 10 from a user, and may communicate the setting information input by the user to a controller 400 of the microneedle device 10 via the cable 30.

Examples of the setting information transferred to the microneedle device 10 may include a final penetration depth of a microneedle, a positive-pressure generation time point (for example, a penetration depth of the microneedle when the positive pressure is generated), an intensity of a pump unit, an operation mode of the microneedle device 10, a time interval for an automatic operation mode, a penetration depth during a needling operation, and the like.

Hereinafter, a configuration of the microneedle device 10 according to an example embodiment of the present disclosure will be described and subsequently, a method of operating the microneedle device 10 according to an example embodiment of the present disclosure will be described.

### 1. Configuration of Microneedle Device According to Example Embodiment of Present Disclosure

### A. Basic Configuration of Microneedle Device

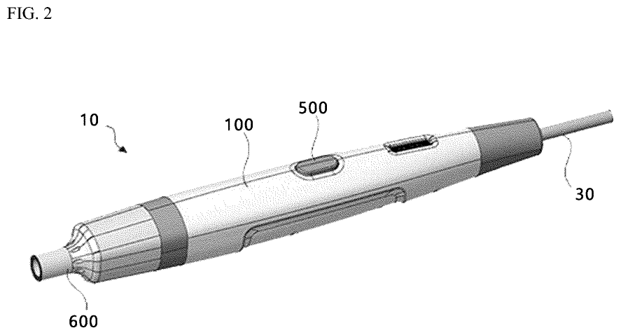

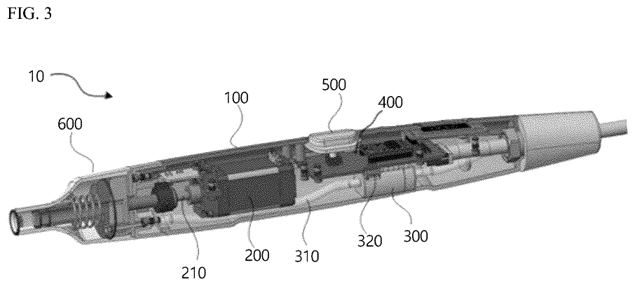

Referring to FIGS. 2 and 3, there are exemplarily illustrated a basic structure of the microneedle device 10 according to an example embodiment of the present disclosure.

As illustrated in FIGS. 2 and 3, the microneedle device 10 according to an example embodiment of the present disclosure may include a housing 100, a driver 200, a pump unit 300, the controller 400 and the like, which are accommodated in the housing 100, and may be configured to operate a needle assembly 600 mounted in front of the microneedle device 10.

According to an example embodiment of the present disclosure, the housing 100 is a part which forms an outer shape of the microneedle device 10, and may be formed of a single member, or may have a structure in which a plurality of members is coupled to each other.

According to an example embodiment of the present disclosure, the needle assembly (microneedle unit) 600 described below may be configured to be coupled to a tip end of the housing 100. The cable 30 described above may be configured to be connected to a rear end of the housing 100.

According to an example embodiment of the present disclosure, the driver 200 is a part which generates and supplies a driving force for operating the microneedle device 10. A linear motor or the like may be used as the driver 200 of the microneedle device 10 according to an example embodiment of the present disclosure.

Unlike a typical electric motor configured to generate a rotational force as a rotor rotates inside a stator, the linear motor is configured so that a mover moves linearly relative to a deployed stator. This makes it possible to easily implement a linear reciprocating motion in a narrow space.

According to an example embodiment of the present disclosure, the pump unit 300 is configured to compress air at a predetermined pressure and supply the same. The pump unit 300 has a function of supplying the compressed air to the needle assembly 600 described below so that effective ingredients such as an effective agent may be easily penetrated into a skin by the compressed air.

According to an example embodiment of the present disclosure, a pneumatic hose 310 may be connected between the pump unit 300 and the needle assembly 600. The compressed air generated in the pump unit 300 may be transferred to the needle assembly 600 via the pneumatic hose 310. A valve 320 may be provided in the pump unit 300. By opening and closing the valve 320, a certain amount of compressed air may be supplied to the needle assembly 600 only when needed.

According to an example embodiment of the present disclosure, the valve 320 provided in the pump unit 300 may be electrically connected to the controller 400. An operation of the valve 320 may be controlled by the controller 400. An example of the valve 320 may include a solenoid valve or the like.

According to an example embodiment of the present disclosure, the controller 400 controls the operation of the microneedle device 10 according to an example embodiment of the present disclosure and may be formed in the form of a printed circuit board (PCB) assembly using a printed circuit board.

According to an example embodiment of the present disclosure, an operation unit 500 may be provided on one side of the housing 100. A user may operate the microneedle device 10 with the operation unit 500.

According to an example embodiment of the present disclosure, the operation unit 500 may be formed in the form of an operation button that the user may press with his/her finger(s). With this configuration, the user may easily operate the operation unit 500 with his/her finger(s) while holding the microneedle device 10 by his/her hand.

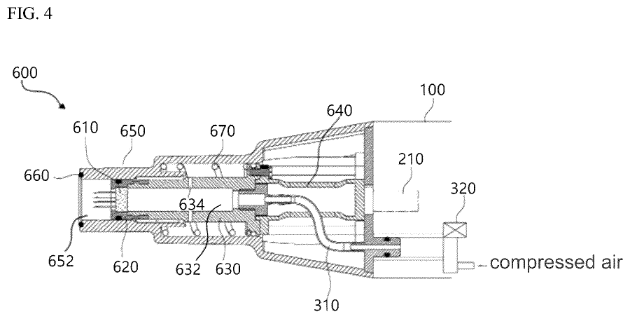

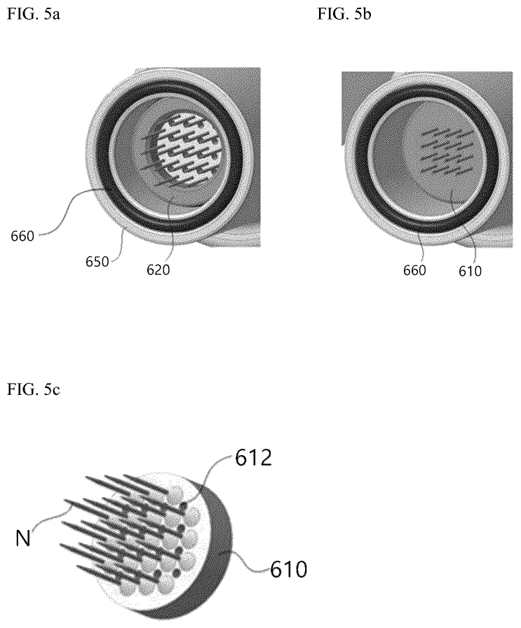

Referring to FIGS. 4 and 5, there are exemplarily illustrated the needle assembly 600 which may be used in the microneedle device 10 according to an example embodiment of the present disclosure.

As illustrated in the figures, the needle assembly 600 may include a plurality of microneedles N which is inserted into a skin of a patient, and may have a function of forming fine holes in the skin of the patient by the microneedles N and efficiently injecting effective ingredients such as effective agents into the skin.

According to an example embodiment of the present disclosure, the plurality of microneedles N may be provided at predetermined intervals in a needle mounting plate 610. The plurality of microneedles N may be mounted and fixed to the needle mounting plate 610 while penetrating the needle mounting plate 610.

According to an example embodiment of the present disclosure, each of the microneedles N may be formed in an approximately tubular structure having a perforated central portion. An inclined bevel portion may be formed at a tip end of each microneedle N so that each microneedle N has a fine-tipped structure in which a tip of a front end portion is sharpened.

According to an example embodiment of the present disclosure, the needle assembly 600 may be formed to have a substantially cylindrical structure and may be coupled to a front side of a plunger 630.

According to an example embodiment of the present disclosure, the plunger 630 of the needle assembly 600 may be formed in a structure having a perforated central portion. The needle mounting plate 610 may be mounted on a front end portion of the plunger 630 and may be configured to cover one end of the open central portion of the plunger 630.

According to an example embodiment of the present disclosure, the needle mounting plate 610 of the needle assembly 600 may be fixed to the front end portion of the plunger 630.

For example, in a case of an example embodiment illustrated in the figures, the needle mounting plate 610 of the needle assembly 600 may be mounted and fixed to the front end portion of the plunger 630 via a fixture 620.

Specifically, in the example embodiment illustrated in the figures, the fixture 620 has a stepped seat portion formed at a rear side thereof. The needle mounting plate 610 is coupled and fixed to the front end portion of the plunger 630 in a state in which the needle mounting plate 610 is coupled to the stepped seat portion.

With this structure, the front end portion of the plunger 630 may be closed by the needle mounting plate 610 and air may flow over both sides of the needle mounting plate 610 via the microneedles N.

According to an example embodiment of the present disclosure, the needle mounting plate 610 may further have discharge holes 612 formed to penetrate the needle mounting plate 610. Further, the air may flow via the discharge holes 612.

According to an example embodiment of the present disclosure, the plunger 630 may be a cylindrical member having an inner hollow portion 632. A rear end portion of the plunger 630 may be fixed to a connection tube 640. A rear end of the connection tube 640 may be connected to a movable rod 210 of the driver 200.

With this structure, when the movable rod 210 reciprocates forward and backward by the driver 200, the connection tube 640 connected to the movable rod 210 also moves forward and backward. As a result, the plunger 630 may reciprocate forward and backward.

According to an example embodiment of the present disclosure, the pneumatic hose 310 connected to the pump unit 300 may be mounted to penetrate the connection tube 640. A front end portion of the pneumatic hose 310 may be connected and fixed to the inner hollow portion 632 of the plunger 630 so that the pneumatic hose 310 is in communication with the plunger 630.

According to an example embodiment of the present disclosure, the pneumatic hose 310 mounted to penetrate the connection tube 640 may be formed to be flexible. Thus, when the connection tube 640 moves, the pneumatic hose 310 may also flexibly move to follow the movement of the connection tube 640, thereby smoothly guiding the movement of the connection tube 640.

According to an example embodiment of the present disclosure, an exhaust hole 634 may be formed in a periphery of the plunger 630. The inner hollow portion 632 of the plunger 630 may be in communication with the outside via the exhaust hole 634.

According to an example embodiment of the present disclosure, the needle assembly 600 may further include a needle cover 650. The needle cover 650 may be coupled and fixed to a front end portion of the housing 100.

According to an example embodiment of the present disclosure, the needle cover 650 may have an inner space 652 open at a central portion thereof. When the microneedle device 10 is used, a tip end of the needle cover 650 may be brought into close contact with the skin of the patient so that the inner space 652 of the needle cover 650 is closed.

To this end, the tip end of the needle cover 650 may include a sealing member 660 (for example, an O-ring). When the microneedle device 10 is used, the sealing member 660 is brought into close contact with the skin to stably close the inner space 652 of the needle cover 650.

According to an example embodiment of the present disclosure, a sealing member (for example, an O-ring) may be further provided between an outer circumferential surface of the plunger 630 and an inner circumferential surface of the needle cover 650 to stably close the inner space 652 of the needle cover 650.

According to an example embodiment of the present disclosure, the plunger 630 (and the needle mounting plate 610 and the connection tube 640 fixed to the plunger 630) may be connected to the needle cover 650 via a spring 670.

For example, in the microneedle device 10 illustrated in the figures, one end of the spring 670 comes into contact with the needle cover 650 to be supported by the needle cover 650, and the other end of the spring 670 comes into contact with one side of the plunger 630 to be supported by the plunger 630. With this configuration, the plunger 630 (and the needle mounting plate 610 and the connection tube 640 fixed to the plunger 630) may be elastically assembled with the needle cover 650 and the housing 100.

### B. Additional Functional Part of Microneedle Device

### (a) RF Energy Transfer Module

The microneedle device 10 according to an example embodiment of the present disclosure may be configured to further have additional functions other than a needling function of forming the fine holes in the skin, thereby further enhancing a skin regeneration and/or treatment effects.

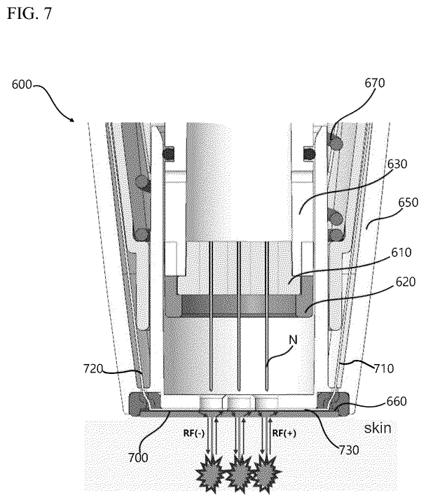

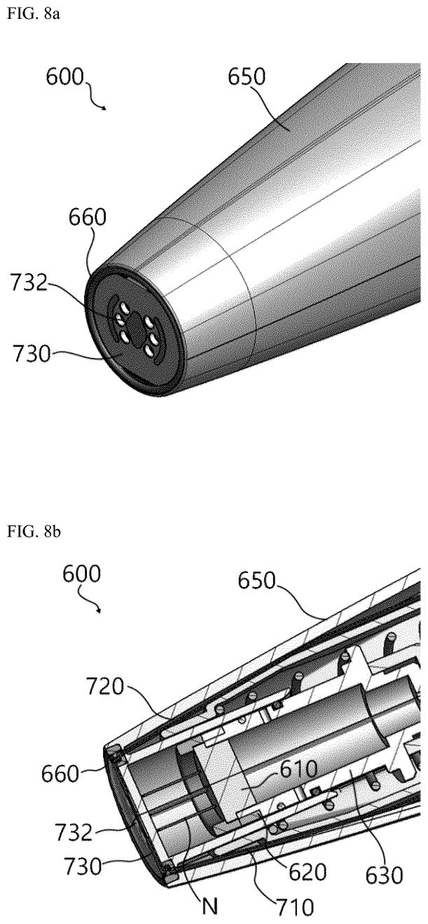

For example, referring to FIGS. 7 and 8, there are exemplarily illustrated an example embodiment in which a RF energy transfer module 700 as an additional functional part is added to the microneedle device 10.

According to an example embodiment of the present disclosure, the RF energy transfer module 700 may include a RF electrode for transferring RF energy to the skin.

According to an example embodiment of the present disclosure, the RF energy transfer module 700 may include one or more RF positive electrodes 710 and one or more RF negative electrodes 720.

According to an example embodiment of the present disclosure, the RF energy transfer module 700 may be configured such that one side thereof is exposed toward the microneedle device 10.

To this end, in an example embodiment illustrated in the figures, the RF energy transfer module 700 may include a contact film 730 provided to face a front end portion of the needle assembly 600. One side (for example, a front end portion) of each of the RF positive electrodes 710 and the RF negative electrodes 720 is electrically connected to the contact film 730.

According to an example embodiment of the present disclosure, the contact film 730 may be formed in a structure covering the front end portion of the needle assembly 600 such that the contact film 730 comes into stable contact with the skin. Further, the contact film 730 may have through-holes 732 formed at positions corresponding to the microneedles N to move the microneedles N therethrough, respectively.

According to an example embodiment of the present disclosure, the other sides (for example, rear end portions) of the RF positive electrodes 710 and the RF negative electrodes 720 may be electrically connected to the controller 400 or the like.

According to an example embodiment of the present disclosure, an operation of the RF energy transfer module 700 may be controlled by the controller 400. As illustrated in FIG. 7, when the microneedles N are inserted into the skin, the fine holes are formed in the skin and the effective agent is injected to the skin via the fine holes. In this state, the RF energy may be transferred to the skin.

With this configuration, in a state in which effective ingredients of the effective agent are applied inward of the skin via the fine holes formed in the skin by the microneedles N, the radio-frequency energy of the RF energy transfer module 700 may be smoothly transferred to deep locations inside the skin.

According to the microneedle device 10 configured as above, in the state in which movement spaces of the effective agent via the fine holes formed in the skin by the microneedles N are formed, the RF energy for RF treatment is applied. Therefore, the RF energy may be easily transferred not only to a surface of the skin in contact with the electrodes but also inward of the skin via the fine holes and the effective agent movement spaces.

In a RF treatment in the related art, the RF energy is applied only to the surface of the skin in a non-invasive manner without forming such fine holes in the skin. Accordingly, a large amount of RF energy needs to be applied to the surface of the skin to transfer the RF energy to deep locations inside the skin. This may cause burns on the surface of the skin. In contrast, in the microneedle device 10 according to an example embodiment of the present disclosure, the RF energy is applied in the state in which the fine holes are formed in the skin by the microneedles N and the movement spaces of the effective agent via the fine holes are secured. Therefore, even if the power of the RF energy is relatively weak, the RF energy may be transferred inward of the skin, thereby performing a desired RF treatment. This makes it possible to easily perform the RF treatment in a more stable manner without causing burns on the surface of the skin.

In addition, the microneedle device 10 according to an example embodiment of the present disclosure is capable of performing the RF treatment simultaneously with the injection of the effective agent by the microneedles N. This makes it possible to maximize effects of the skin regeneration and/or the skin treatment by a synergistic action of the effective agent treatment and the RF treatment.

### (b) Light Irradiation Module

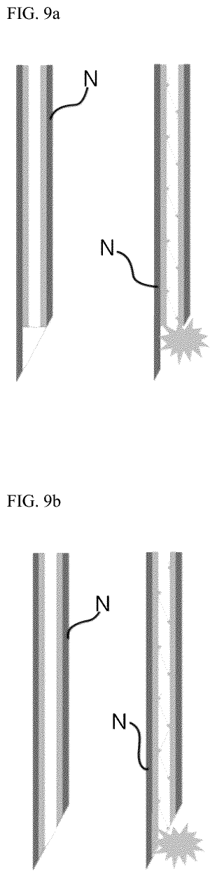

Referring to FIGS. 8A to 9B, there are exemplarily illustrated, as Modifications of the above-described example embodiments, a microneedle device 10 which further includes a light irradiation part 800 for light irradiation.

According to an example embodiment of the present disclosure, the light irradiation part 800 may be configured to irradiate light generated from a light source (not illustrated) forward of the microneedle device 10 via a through-hole formed at a central portion of each of the microneedles N.

According to an example embodiment of the present disclosure, a laser, a light-emitting device (LED) or the like may be used as the light source of the light irradiation part 800.

According to an example embodiment of the present disclosure, in order to more smoothly perform the light irradiation by the light irradiation part 800, an inner circumferential surface of the through-hole formed at the central portion of each microneedle N may be coated with a material having a relatively high reflectance, or an optical fiber 810 may be inserted into the through-hole.

According to an example embodiment of the present disclosure, in the case in which the optical fiber 810 is inserted into the through-hole at the central portion of each microneedle N, as illustrated in FIG. 9, the optical fiber 810 may be formed in a tubular structure in which the central portion is open, or a solid structure having both a core and a cladding.

With this configuration, the light emitted from the light irradiation part 800 may be directly irradiated to a target location via the fine holes formed in the skin by the microneedles N. This makes it possible to efficiently perform the light irradiation without causing damage on the surface of the skin.

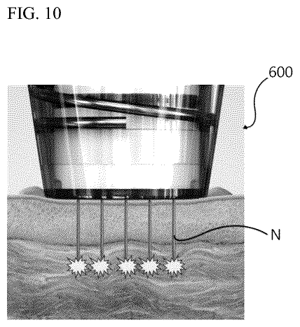

For example, in order to transfer energy to the skin by irradiating laser deeply into the skin, a relatively strong energy of laser needs to be irradiated. This may cause matters such as bums on the surface of the skin. However, the microneedle device 10 according to an example embodiment of the present disclosure may directly irradiate the light to desired locations inside the skin via the through-holes of the microneedles N (see FIG. 10). This makes it possible to stably perform the light irradiation with the light of an appropriate intensity.

According to an example embodiment of the present disclosure, the light irradiation part 800 may include light sources configured to emit light having a plurality of wavelengths. The light irradiation part 800 may irradiate lights of different wavelengths corresponding to different depths inside the skin to perform an optimal light irradiation.

In addition, the above-described light irradiation module may be changed to be applied to other various forms which require light energy.

As an example, in a case in which an effective agent that needs to act on a specific lesion or at a specific skin depth is used, the effective agent may be encapsulated with a substance whose morphology changes (for example, dissolves) at a specific wavelength (or specific temperature). The effective agent thus encapsulated may be injected to the skin by the microneedles N. Thereafter, the effective agent may work by being irradiated with a light of an appropriate wavelength (or by being supplied with energy that may induce an increase in temperature) at a treatment location. This makes it possible to precisely perform the treatment at the specific lesion or specific skin depth.

Further, according to an example embodiment of the present disclosure, the microneedle device 10 may be further include a cooling means as an option to prevent the temperature from being increased due to the light irradiation.

### 2. Basic Operating Principle of Microneedle Device According to Example Embodiment of Present Disclosure

A basic operating principle of the microneedle device 10 configured as above according to an example embodiment of the present disclosure is as follows.

Prior to specifically describe the operating principle, the microneedle device 10 according to an example embodiment of the present disclosure will be conceptually described with a focus on obvious differences from a technology in the related art.

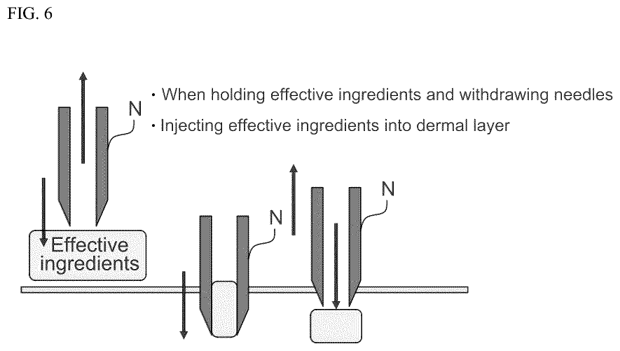

As illustrated in FIG. 6, the microneedle device 10 according to an example embodiment of the present disclosure uses a method of applying effective ingredients such as an effective agent to the skin and injecting the effective ingredients into a dermal layer of the skin with the microneedles N instead of using an injection liquid (for example, FIG. 6 illustrates an example embodiment in which hollow microneedles N are used).

As described above, the microneedle device 10 according to an example embodiment of the present disclosure does not use the injection liquid. Therefore, the present disclosure is not regulated by medical treatment laws. Thus, the present disclosure may be applied to various skin boosters other than limited effective agents.

As illustrated in FIG. 6, effective ingredients may be injected to the skin by applying a skin booster as the effective ingredients to the skin, followed by bring each microneedle N close to the skin so that the effective ingredients is received in the central hollow of each microneedle N, followed by penetrating each microneedle N into the skin, followed by blowing compressed air into the central hollow of each microneedle N at the time of penetrating each microneedle N into the skin or after each microneedle N penetrates into the skin, followed by removing each microneedle N from the skin.

Next, a needling operation of the microneedle device 10 according to an example embodiment of the present disclosure will be described in detail.

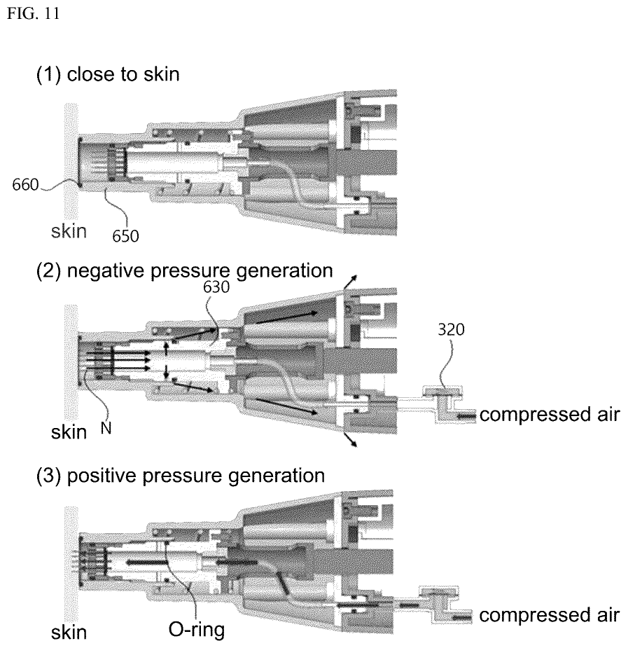

As illustrated in FIG. 11, a method of operating the microneedle device 10 according to an example embodiment of the present disclosure may include a skin close-contacting operation, a negative-pressure generating operation and a positive-pressure generating operation.

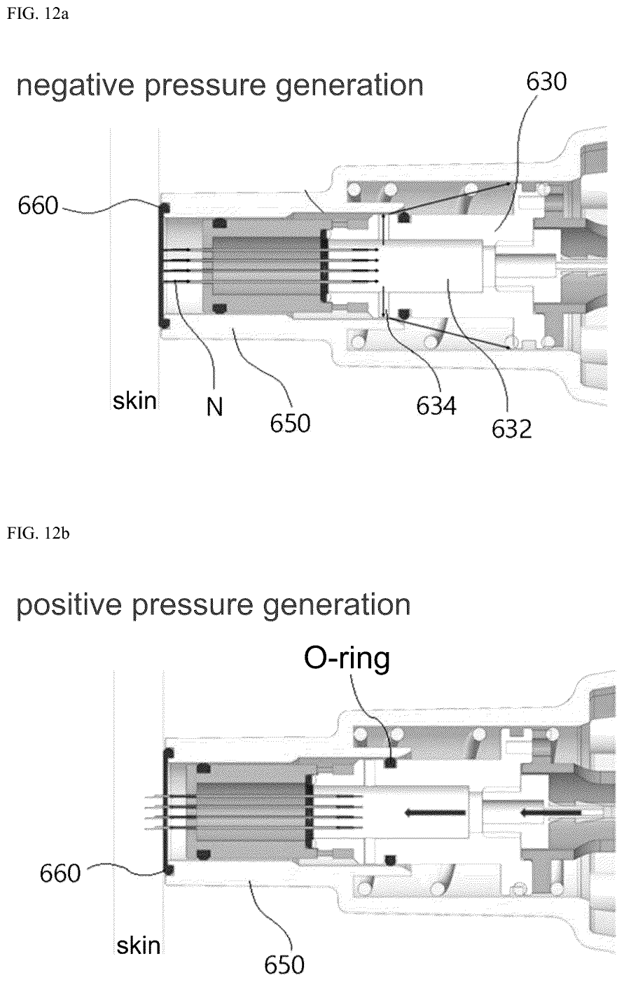

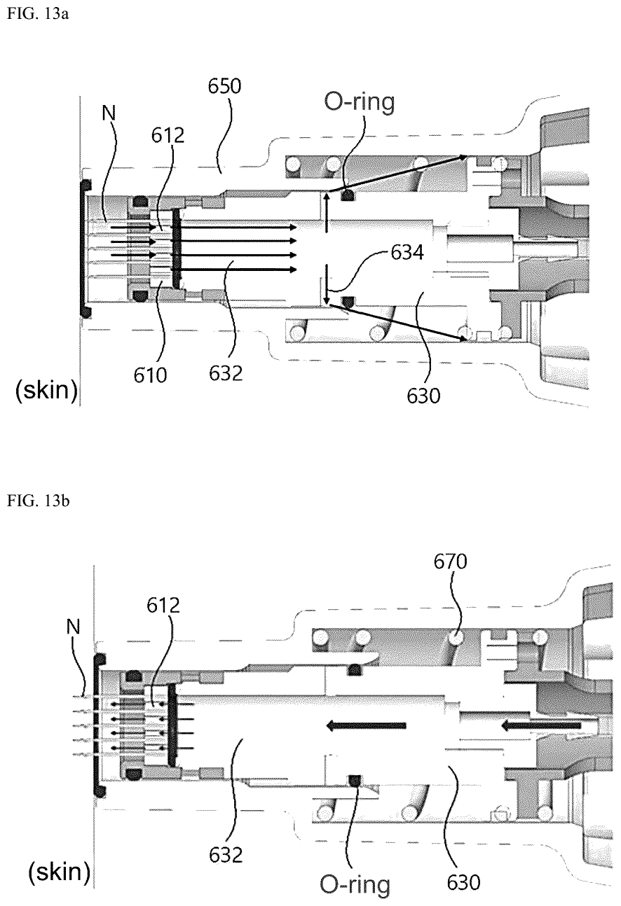

Hereinafter, the method of operating the microneedle device 10 illustrated in FIG. 11 will be described with reference to an example embodiment of the microneedles N of a hollow type illustrated in FIGS. 12A and 12B.

First, the skin close-contacting operation is an operation of bringing the tip end of the needle cover 650 of the needle assembly 600 into close contact with the skin in a state in which effective ingredients is applied to the skin, and forming a closed space inside the needle cover 650.

According to an example embodiment of the present disclosure, in the skin close-contacting operation, when the sealing member 660 located at the front end portion of the needle cover 650 is brought into close contact with the skin, the inner space 652 of the needle cover 650 may be closed to form a closed space.

At this time, the microneedles N are in a home position state. The expression” home position state” means a state in which the microneedles N are positioned at an interval with respect to the front end portion of the needle cover 650. In the home position state, the pump unit 300 is operated to generate the compressed air, but the supply of the compressed air is blocked by the valve 320.

The negative-pressure generating operation is an operation of discharging air existing in the closed space between the skin and the front end of the plunger 630 via the exhaust hole 634 of the plunger 630 while moving the plunger 630 forward by the driver 200, and forming a negative pressure in the closed space as the inner space 652 of the needle cover 650.

In this case, the air in the closed space as the inner space 652 of the needle cover 650 moves to the inner hollow portion 632 of the plunger 630 via the inner hollow of each microneedle N and is discharged to the outside via the exhaust hole 634.

As described above, when the negative pressure is generated in the closed space as the inner space 652 of the needle cover 650, the effective ingredients applied to the skin may be easily flown into the inner hollow of each microneedle N.

At this time, the compressed air generated by the pump unit 300 remains blocked by the valve 320.

The positive-pressure generating operation is an operation of further moving the plunger 630 forward such that the microneedles N penetrate the skin while holding the effective ingredients, and opening the valve 320 to supply the compressed air generated from the pump unit 300 so that the effective ingredients are injected into the skin.

According to an example embodiment of the present disclosure, the plunger 630 may be moved backward and the microneedles N may be retracted from the skin to further supply the compressed air for several seconds, so that the closed space is kept at a positive pressure.

A sequence of operations as described above may provide the same effects as those of directly injecting effective ingredients such as effective agents into the skin with needles. This makes it possible to inject the effective ingredients such as effective agents into the skin in a more smooth and efficient manner.

In this case, the spring 670 may perform a function of applying an elastic force so that the plunger 630 (and the needle mounting plate 610, the connection tube 640 and the like, which are fixed to the plunger 630) may smoothly return to the home position.

Alternatively, in the microneedle device 10 according to an example embodiment of the present disclosure, the microneedles N may have a solid structure as illustrated in FIGS. 12A and 12B, instead of the hollow structure described above.

For example, as illustrated in FIGS. 12A and 12B, the microneedles N may be configured to have a solid structure whose interior is filled. The needle mounting plate 610 may be formed in a structure in which one or more discharge holes 612 are provided between the microneedles N.

The microneedles N having such a solid structure may be operated as illustrated in FIGS. 12A and 12B. An operation of the microneedles N having the solid structure is substantially similar to that of the microneedles N having the hollow structure as described with reference to FIG. 11, and therefore, only differences between the operation of the microneedles N having the solid structure and the operation of the microneedles N having the hollow structure will be additionally described below.

That is, when the tip end of the needle cover 650 is brought into close contact with the skin in the state in which the effective ingredients are applied onto the skin, the closed space is formed between the skin and the needle cover 650 and the air existing in the closed space moves to the inner hollow portion 632 of the plunger 630 and is discharged via the discharge hole 612. As a result, the closed space is kept at the negative pressure as illustrated in FIG. 12A.

In this state, as illustrated in FIG. 12B, the plunger 630 is moved forward and the microneedles N penetrates the skin. When the compressed air is supplied to the inner hollow portion 632 of the plunger 630, the closed space is kept at the positive pressure so that the effective ingredients applied on the skin are injected into the skin. The effective ingredients may be injected via gaps between outer circumferential surfaces of the microneedles N and the skin. Thereafter, when the plunger 630 is moved backward and the microneedles N are retracted to further supply the compressed air, the closed space remains kept at the positive pressure, so that the effective ingredients are smoothly and efficiently injected and filled into sites from the microneedles N are retracted.

A sequence of operations as described above may provide the same effects as those of directly injecting effective ingredients such as effective agents into the skin with needles. This makes it possible to inject the effective ingredients such as effective agents into the skin in a more smooth and efficient manner.

Next, a method of transferring the RF energy in the microneedle device 10 according to an example embodiment of the present disclosure will be described.

Referring to FIG. 7, there is exemplarily illustrated how the RF energy is transferred in the microneedle device 10 according to an example embodiment of the present disclosure.

According to an example embodiment of the present disclosure, as illustrated in FIG. 7, the RF energy may be transferred in the state in which the fine holes are formed in the skin by the needling operation and the effective ingredients such as effective agents are injected into the skin via the fine holes.

That is, the transfer of the RF energy into the skin may be performed by applying the radio frequency with the RF positive electrode 710 and the RF negative electrode 720 after the needling operation as described above.

The microneedle device 10 according to an example embodiment of the present disclosure is configured to apply the radio frequency in the state in which the fine holes are formed in the skin of the user by the needling operation and the effective ingredients such as effective agents are injected via the fine holes. Thus, as illustrated in FIG. 7, the RF energy may be stably transferred to deep locations inside the skin via the fine holes formed in the skin and the effective agents injected via the fine holes.

Therefore, even if RF energy of a relatively weak intensity is applied, the RF energy may be stably supplied to deep locations inside the skin to perform the RF treatment. This makes it possible to stably perform the RF treatment without causing burns on the skin or the like.

Next, a method of perform the light irradiation in the microneedle device 10 according to an example embodiment of the present disclosure will be described.

Referring to FIG. 10, there is exemplarily illustrated how the microneedle device 10 according to an example embodiment of the present disclosure irradiates a desired site with light using the light irradiation part 800.

As illustrated in the figure, the microneedle device 10 according to an example embodiment of the present disclosure may be configured such that the light generated from the light irradiation part 800 is irradiated forward via a through-hole formed in each microneedle N of a tubular shape in the state in which the microneedles N are inserted into the skin.

According to an example embodiment of the present disclosure, the light irradiation part 800 may be controlled to irradiate the light in the state in which the microneedles N are inserted into the skin by a preset depth. The depth to which the light is irradiated may be set by the user.

The microneedle device 10 according to an example embodiment of the present disclosure is configured such that the light is irradiated into the skin via the through-hole formed in the central portion of each microneedle N in the state in which the microneedles N are inserted into the skin. This makes it possible to directly irradiate a desired site inside the skin with the light, thereby easily and efficiently perform the light irradiation even with light of a relatively weak intensity.

### 3. Control of Positive Pressure Generation by Microneedle Device According to Example Embodiment of Present Disclosure

As described above, the microneedle device 10 according to an example embodiment of the present disclosure is configured to generate the positive pressure using the pump unit 300 and the valve 320, rather than generating the positive pressure through a change in volume by the movement of the plunger 630. Thus, it is possible to control a time point at which the positive pressure is generated independent of the movement of the plunger 630.

That is, the microneedle device 10 according to an example embodiment of the present disclosure may control the time point of the generation of the positive pressure independent of the movement of the plunger 630 by controlling the operation of the pump unit 300 and time points at which the valve 320 are open and closed.

Accordingly, depending on a condition of the skin to be treated, it is possible to optimize a profile in which the effective ingredients are injected into the skin (a profile in which the effective ingredients are injected to correspond to different depths of the skin). For example, the effective ingredients may be injected uniformly regardless of the different depths of the skin. Alternatively, the effective ingredients may be injected intensively at a specific depth. Such an action may be further maximized by adjusting power of the pump unit 300.

That is, by generating and maintaining a predetermined positive pressure as soon as the microneedles N begin to penetrate the skin (or before the penetration), it is possible to uniformly inject the effective ingredients regardless of the different depths of the skin. Alternatively, when the microneedles N reaches the specific depth, the compressed air of a relatively high pressure may be blown into the microneedles N to intensively inject the effective ingredients into the specific depth. In the case, the specific depth may be different from that at which the microneedles N will finally penetrate the skin (hereinafter referred to as “final penetration depth”).

As described above with reference to FIGS. 1 and 3, the user may input, as the setting information, a time point at which a desired positive-pressure is generated (the penetration depth of the microneedle at the time of generating the positive pressure, that is, the specific depth) and a desired power of the pump unit 300, with the user interface 25 of the display device 20. The setting information is transmitted to the controller 400 of the microneedle device 10. The controller 400 controls the opening/closing of the valve 320, the power of the pump unit 300 and the like based on the setting information.

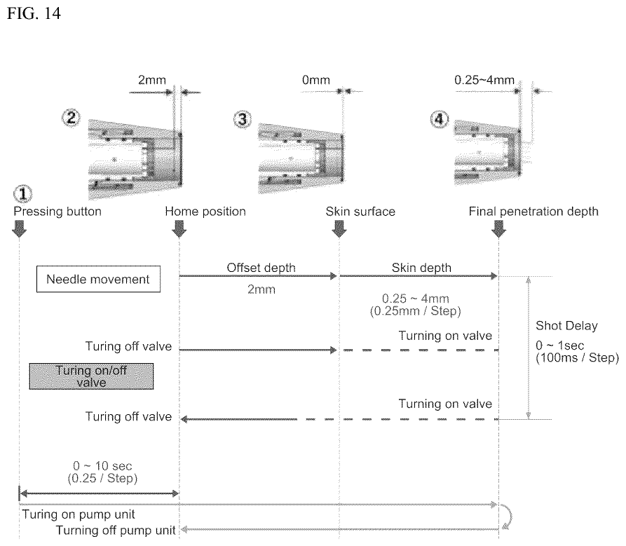

FIG. 14 illustrates a procedure of controlling the operation of the pump unit 300 and the opening/closing of the valve 320 according to an example embodiment of the present disclosure.

When the operation button of the operation unit 500 is pressed by the user, the microneedle device 10 starts to operate.

In an initial state of the microneedle device 10 before the user presses the operation button of the operation unit 500, the pump unit 300 remains turned off and the valve 320 also remains turned off (the valve 320 remains closed).

When the user presses the operation button of the operation unit 500, the pump unit 300 is turned on to generate the compressed air. The flow of the compressed air is blocked by the valve 320. At this time, the microneedles N are in the home position state (for example, the microneedles N are offset inward from the tip end of the needle cover 650).

When the operation button of the operation unit 500 is pressed by the user and a predetermined time period (about 10 seconds or less) elapses, the microneedles N starts to move forward. In the linear motor, since a movement distance of the movable rod 210 per pulse is set in advance, the movement distance of the movable rod 210, that is, a movement distance of the microneedles N may be determined based on the number of pulses.

When the microneedles N are moved by an offset distance, the microneedles N come into contact with the skin, and subsequently penetrate the skin. In the case, the offset distance may be set in a range of more than zero to 7 mm or less. The offset distance may be set according to a configuration of the needle assembly (microneedle unit) and user's requirement. For example, when the offset distance is set in increments of 0.25 mm in the range of more than zero to 1 mm or less, the offset distance may be set to 0.25 mm, 0.5 mm, 0.75 mm, and 1 mm. Even in the remaining range of more than 1 mm to 7 mm or less, the offset distance may be set in the same manner as the above.

In the microneedle device 10 according to an example embodiment of the present disclosure, the positive pressure is generated by turning (opening) the valve 320 on. That is, a time point at which the valve 320 is turned on may be the positive-pressure generation time point.

The profile in which the effective ingredients are injected to correspond to different depths of the skin may vary depending on the positive-pressure generation time point, a magnitude of the positive pressure, a type of effective ingredients (that is, skin booster) or the like. Further, a skin state at a site to be treated (for example, a position or state of an epidermal layer, a position or state of a dermal layer, or the like) varies depending on a person to be treated and a skin site of the person (forehead site, eye site or the like.).

In the microneedle device 10 according to an example embodiment of the present disclosure, the user may set the positive-pressure generation time point or the magnitude of the positive pressure in consideration of the type of skin booster, a skin condition of a site to be treated, and the like to obtain a profile in which desired effective ingredients are injected.

For example, when wanting effective ingredients to be intensively injected at a specific depth of 3 mm in consideration of a position of the epidermal layer or a position of the dermal layer at the skin site to be treated, the user may set the positive-pressure generation time point or the magnitude of the positive pressure corresponding thereto. The specific depth of 3 mm mentioned as above is merely an example. The specific depth may be set in the range of more than zero to 7 mm or less. For example, when the specific depth is set in increments of 0.25 mm in the range of more than zero to 1 mm or less, the specific depth may be set to 0.25 mm, 0.5 mm, 0.75 mm, and 1 mm. Even in the remaining range of more than 1 mm to 7 mm or less, the specific depth may be set in the same manner as the above.

According to an example embodiment of the present disclosure, the positive-pressure generation time point may be set to correspond to a penetration depth of the microneedles N at the positive-pressure generation time point so that the user may intuitively set the positive-pressure generation time point. For example, in a case in which the user sets to a depth of 3 mm as the positive-pressure generation time point (the penetration depth of the microneedles N at the positive-pressure generation time point), when the penetration depth of the microneedles N is 3 mm, the valve 320 is open to generate the positive pressure.

Further, according to the profile in which the desired effective ingredients are injected, the positive-pressure generation time point may be set while the microneedles N move forward, or may be set while the microneedles N move backward.

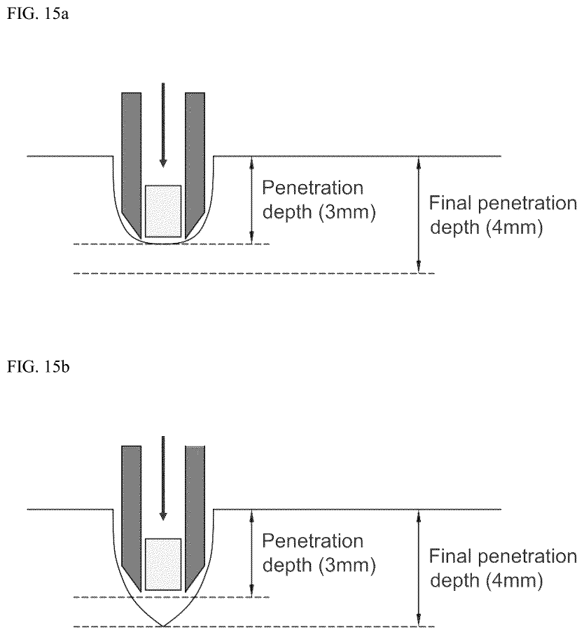

For example, FIG. 15A illustrates a state in which, when the final penetration depth is 4 mm, the microneedle N moves forward by 3 mm. FIG. 15B illustrates a state in which, when the final penetration depth is 4 mm, the microneedle N penetrates by 4 mm, which is the final penetration depth, and subsequently move backward by 1 mm. FIGS. 15A and 15B illustrate states in which, when the penetration depth of the microneedle N is 3 mm, shapes formed in the skin by the penetration of the microneedle N are different from each other. In this case, the profile in which the effective ingredients are injected may vary.

For example, as illustrated in FIG. 15B, in the case in which the positive pressure is generated while the microneedle N penetrates by the final penetration depth of 4 mm and subsequently moves backward by 1 mm, the effective ingredients may be easily injected to a site from which the microneedle N is just withdrawn. In particular, the effective ingredients may be intensively injected around the depth of 3 mm.

In addition, the positive pressure may be maintained for a predetermined period of time even after the microneedle N has completely withdrawn from the skin. With this configuration, even after the microneedle N has completely withdrawn from the skin, the effective ingredients may be more easily injected to the site in the epidermal layer from which the microneedle N is withdrawn.

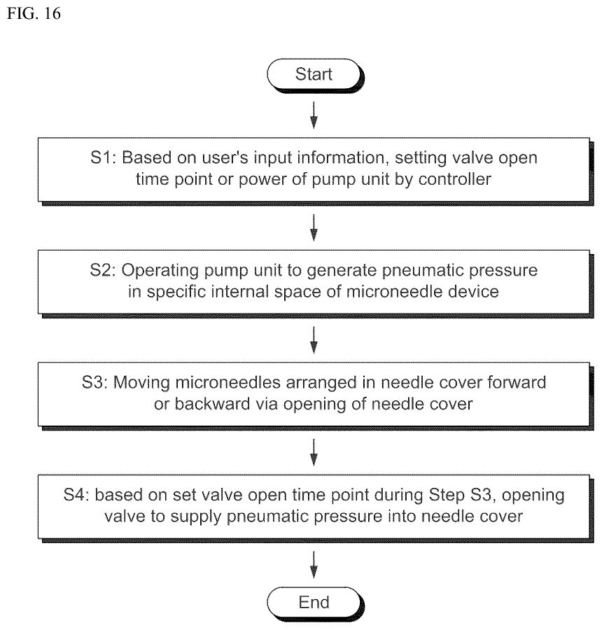

FIG. 16 is a flowchart exemplarily explaining a method of injecting effective ingredients into the skin using the microneedle device according to an example embodiment of the present disclosure.

As illustrated in FIG. 16, based on information input by the user (which is input using the display device and transmitted to the controller), the controller 400 may set a time point at which the valve is open or the power of the pump unit (step S1). Subsequently, when the user presses the operation button of the operation unit 500, the pump unit 300 is operated to generate a pneumatic pressure in a specific internal space of the microneedle device (step S2). The microneedles N arranged in the needle cover 650 move forward or backward via the opening of the needle cover 650 (step S3). When a time point at which the valve is set to be open reaches during the operation of Step S3, the valve 430 is open to supply the pneumatic pressure generated by the pump unit 300 into the needle cover 650 (step S4).

### 4. Operation Mode of Microneedle Device according to Example Embodiment of Present Disclosure

### A. Manual Mode and Automatic Mode

As illustrated in FIG. 14, the microneedle device 10 according to an example embodiment of the present disclosure starts to operate when the user presses the operation button of the operation unit 500. That is, when the user presses the operation button, operation start information input by the user is transferred to the controller 400. The controller 400 controls the microneedles N to move forward and backward at an initial home position and return to the home position again, which is referred to one round of needling operation. Further, the controller 400 controls the pump unit 300 and the valve 320 to be turned on and off during the needling operation according to a preset condition.

According to an example embodiment of the present disclosure, the microneedle device 10 may be set to perform the above-described operations each time the user presses the operation button of the operation unit 500 (in a manual mode). Alternatively, the microneedle device 10 may be set to perform the above-described operations automatically every set time interval when the user presses the operation button of the operation unit 500 (in an automatic mode).

As an example, the time interval in the automatic mode may be set in a range of 100 to 1,000 milliseconds (msec). For example, the time interval may be set to any one of 100, 200, 300, 400, 500, 600, 700, 800, 900, and 1,000 msec.

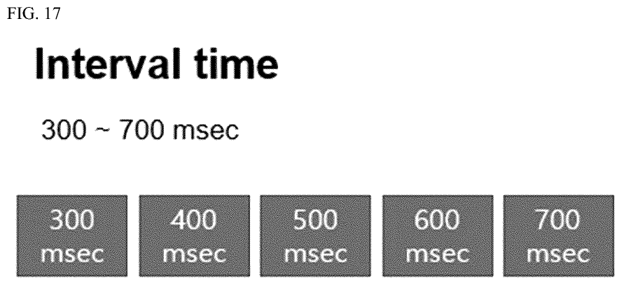

The display device 20 of the microneedle device 10 may provide the user interface 25 for receiving such setting information from the user. The setting information input from the user (that is, information about whether to operate in the manual mode or operate in the automatic mode, and time interval information in the case of the automatic mode) may be transmitted to the controller 400 of the microneedle device 10 via the cable 30. FIG. 17 exemplarily illustrates an example of the user interface for receiving the time interval information in a case where the time interval is set in a range of 300 to 700 msec.

### B. Stacking Mode

According to an example embodiment of the present disclosure, the penetration depths of the microneedles N in the automatic mode may be set to be different from each other according to the needling operation. For example, the penetration depths of the microneedles N may be set for each needling operation of a plurality of predetermined consecutive needling operations, which is referred to as a stacking mode. The stacking mode may be performed repeatedly.

According to an example embodiment of the present disclosure, in the stacking mode, the needling operation may be repeatedly performed twice or more times at the same skin position or at different time intervals.

According to an example embodiment of the present disclosure, the number of needling operations in the stacking mode may be variously set. For example, the number of needling operations may be selected in a range of two to ten times.

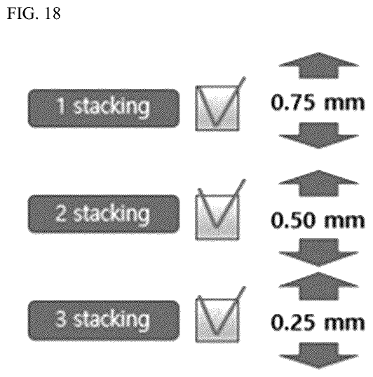

FIG. 18 exemplarily illustrates an example of the user interface for setting the penetration depth for each needling operation when the number of needling operations in the stacking mode is three. For the sake of convenience in description, in this specification, each needling operation in one stacking mode is referred to as “stacking.” That is, in one stacking mode, a first needling operation will be referred to as “first stacking,” and a second needling operation will be referred to as “two stacking.”

As illustrated in FIG. 18, the penetration depth may be set for each stacking (needling). The penetration depth for each stacking may be set to be identical to that in a previous stacking, and may be set to be different from that in the previous stacking.

Referring to FIG. 18, the first stacking is set to perform the needling operation by the penetration depth of 0.75 mm. In this case, the user may increase or decrease the set penetration depth by clicking an upward arrow or a downward arrow. That is, at the time of the first stacking, the microneedles N move forward at the home position by the penetration depth of 0.75 mm and subsequently return to the home position. In the second stacking, the needling operation is set to be performed by the penetration depth of 0.50 mm. When the time interval is set to 100 msec, the second stacking is performed after 100 msec from when the first stacking is completed. That is, the microneedles N, which has been returned to the home position, move forward by the penetration depth of 0.50 mm and subsequently move backward to return to the home position. Similarly, the third stacking is performed after 100 msec from when the second stacking is completed. That is, the microneedles N move forward by the penetration depth of 0.25 mm and subsequently move backward to return to the home position. The stacking mode as described above may be repeatedly performed. For example, the first stacking may be performed again after 100 msec from when the third stacking is completed.

When the user presses the operation button of the operation unit 500 once in the stacking mode, the microneedles N may penetrate into the same skin position several times, which makes it possible to treat several layers in the skin (for example, a papillary layer, a plexiform layer and the like) at once by a mechanism of microneedling and the injection of the effective ingredients (effective agents and the like). That is, the entire skin layer may be treated.

For example, in a case of a general mode other than the stacking mode, 100 rounds of needling operations are assumed to be performed at each of two different penetration depths (first and second penetration depths) in the entire facial skin. In this case, the penetration depth of the microneedle device is first set to the first penetration depth and the microneedle device needs to perform 100 rounds of needling operations while moving on the entire facial skin, and subsequently, the penetration depth of the microneedle device is set to the second penetration depth and the microneedle device needs to perform 100 rounds of needling operations while moving again along the same path on the entire facial skin. In contrast, in the case of the stacking mode, the first stacking may be set to performed by the first penetration depth and the second stacking may be set to performed by the second penetration depth. Thereafter, the microneedle device may perform 200 rounds of needling operations (that is, the stacking mode may be repeated 100 times) while moving once on the entire facial skin.

Although the present disclosure has been described above in terms of specific items such as detailed constituent elements as well as the limited example embodiments and the drawings, they are merely provided to help more general understanding of the present disclosure, and the present disclosure is not limited to the above example embodiments. Various modifications and changes could have been realized by those skilled in the art to which the present disclosure pertains from the above description.

Therefore, the spirit of the present disclosure need not to be limited to the above-described example embodiments, and in addition to the appended claims to be described below, and all ranges equivalent to or changed from these claims need to be said to belong to the scope and spirit of the present disclosure.

### EXPLANATION OF REFERENCE NUMERALS

10: Microneedle device
25: User interface
100: Housing
210: Movable rod
310: Pneumatic hose
20: Display device
30: Cable
200: Driver
300: Pump unit
320: Valve
400: Controller
600: Needle assembly
620: Fixture
640: Connection tube
660: Sealing member
500: Operation unit
610: Needle mounting plate
630: Plunger
650: Needle cover
670: Spring
700: RF energy transfer module
710: RF positive electrode
720: RF negative electrode
730: Contact film
800: Light irradiation module
810: Optical fiber

1. A microneedle device for forming fine holes in a skin to inject effective ingredients applied on the skin into the skin via the fine holes, the microneedle device comprising:
a housing;
a driver configured to generate a driving force for operating a plurality of microneedles;
a needle assembly including the plurality of microneedles;
a controller configured to control an operation of the microneedle device; and
a radio-frequency (RF) energy transfer module configured to transfer radio frequency energy to the skin,
wherein the RF energy transfer module includes an RF positive electrode and an RF negative electrode, and
wherein one side of the RF energy transfer module is exposed forward of the microneedle device.

2. The microneedle device of claim 1, wherein the RF energy transfer module includes a contact film at a front end portion of the needle assembly, and
wherein one side of the RF positive electrode and one side of the RF negative electrode are electrically connected to the contact film.

3. The microneedle device of claim 1, wherein, in a state in which the plurality of microneedles is inserted into the skin to form the fine holes and effective agents are injected to the skin via the fine holes, the radio frequency energy is transferred to the skin by the RF positive electrode and the RF negative electrode.

4. A microneedle device for forming fine holes in a skin to inject effective ingredients applied on the skin to the skin via the fine holes, the microneedle device comprising:
a housing;
a driver configured to generate a driving force for operating a plurality of microneedles;
a needle assembly provided with the plurality of microneedles;
a controller configured to control an operation of the microneedle device; and
a light irradiation module configured to irradiate the skin with light via the plurality of microneedles,
wherein each of the plurality of microneedles is formed in a tubular structure having a through-hole at a central portion thereof, and
wherein the light irradiation module is configured to irradiate the light forward of the microneedle device via the through-hole formed in the central portion of each of the plurality of microneedles.

5. The microneedle device of claim 4, wherein an inner circumferential surface of the through-hole formed in the central portion of each of the plurality of microneedles is coated with a material having a relatively high reflectance.

6. The microneedle device of claim 4, wherein an optical fiber is inserted into the through-hole formed in the central portion of each of the plurality of microneedles.

7. The microneedle device of claim 4, wherein the light irradiation module is configured to irradiate the skin with the light in a state in which the plurality of microneedles is inserted into the skin.

8. The microneedle device of claim 4, wherein a light source of the light irradiation module is configured to emit light of a plurality of wavelengths.

9. The microneedle device of claim 4, wherein the light irradiation module is configured to irradiate light of different wavelengths according to penetration depths of the plurality of microneedles.

10. The microneedle device of claim 1, further comprising:
a pump unit configured to generate compressed air and supply the compressed air into the needle assembly; and
a valve configured to open and close a pneumatic hose provided between the pump unit and the needle assembly.

11. The microneedle device of claim 10, wherein the valve is open and closed under a control of the controller,
wherein, when a tip end of the needle assembly is brought into close contact with the skin, an interior of the needle assembly is closed to form a closed space, and
wherein, when the valve is open, the compressed air is supplied from the pump unit to the interior of the needle assembly so that a positive pressure is generated in the closed space of the needle assembly.

12. The microneedle device of claim 11, wherein the controller is configured to control the opening and closing of the valve based on information input from a user.

13. The microneedle device of claim 1, wherein the controller is configured to control a needling operation in which the plurality of microneedles is penetrated into the skin and subsequently return to original positions thereof; and
wherein the controller is configured to control the microneedle device in a stacking mode in which a penetration depth is set for each needling operation.

14. The microneedle device of claim 13, wherein the stacking mode is set such that the needling operation is repeated twice or more times at a same skin position or at different time intervals, and
wherein, in the stacking mode, the needling operation is performed at a same penetration depth or different penetration depths.

15. A microneedle system, comprising:
the microneedle device of claim 1; and
a display device which is in communication with the controller of the microneedle device, and configured to provide a user interface for receiving setting information about the operation of the microneedle device from a user and transmit the setting information input from the user to the controller of the microneedle device.

16. The microneedle device of claim 4, further comprising:
a pump unit configured to generate compressed air and supply the compressed air into the needle assembly; and
a valve configured to open and close a pneumatic hose provided between the pump unit and the needle assembly.

17. The microneedle device of claim 4, wherein the controller is configured to control a needling operation in which the plurality of microneedles is penetrated into the skin and subsequently return to original positions thereof; and
wherein the controller is configured to control the microneedle device in a stacking mode in which a penetration depth is set for each needling operation.

18. A microneedle system, comprising:
the microneedle device of claim 4; and
a display device which is in communication with the controller of the microneedle device, and configured to provide a user interface for receiving setting information about the operation of the microneedle device from a user and transmit the setting information input from the user to the controller of the microneedle device.
